(19) United States
(12) Patent Application Publication
(10) Pub. No.: US 2024/0277399 A1
(43) Pub. Date: Aug. 22, 2024

(54) RF WIRE CONNECTION IN DILATOR

(71) Applicant: Boston Scientific Scimed, Inc., Maple Grove, MN (US)

(72) Inventor: Spencer Fodness-Bondhus, Columbia Heights, MN (US)

(21) Appl. No.: 18/437,600

(22) Filed: Feb. 9, 2024

Related U.S. Application Data
(60) Provisional application No. 63/447,595, filed on Feb. 22, 2023.

(51) Int. Cl. A61B 18/14; A61B 18/00; A61B 18/12; A61M 29/00

(52) U.S. Cl. CPC A61B 18/14; A61B 18/1206; A61M 29/00; A61B 2018/00077; A61B 2018/00083; A61B 2018/00351; A61B 2018/00601; A61B 2018/00708; A61B 2018/1422; A61B 2018/1435; A61B 2018/1475; A61M 2205/054

(57) ABSTRACT

A transseptal access system is disclosed. The system includes a radiofrequency perforation device having a proximal portion and a distal portion. The proximal portion has an electrically insulated outer surface, the distal portion is electrically conductive and terminates at a functional tip, and at least part of the distal portion is uninsulated. A tubular dilator is further disclosed comprising an elongated body terminating in a distal tip, a hub at the proximal end of the body, and a dilator lumen extending through the hub and the body. The dilator lumen is dimensioned to slidingly receive the radiofrequency perforation device. An electrical contact element disposed within the hub adjacent to the dilator lumen is also disclosed. When the radiofrequency perforation device is in a first position within the dilator lumen, the electrical contact element is in electrical engagement with the distal portion of the radiofrequency perforation device.

### CROSS REFERENCE TO RELATED APPLICATIONS

The present application claims the benefit of U.S. Provisional Application No. 63/447,595, entitled “RF WIRE CONNECTION IN DILATOR,” filed Feb. 22, 2023, the disclosure of which is incorporated herein in its entirety.

### TECHNICAL FIELD

The present invention relates generally to methods and devices usable to deliver energy within the body of a patient. More specifically, the present invention is concerned with a radiofrequency perforation apparatus.

### BACKGROUND

Devices currently exist for creating a puncture, channel, or perforation within a tissue located in a body of a patient. One such device is the Brockenbrough™ Needle, which is commonly used to puncture the atrial septum of the heart. This device is a stiff elongated needle, which is structured such that it may be introduced into a body of the patient via the femoral vein and directed towards the heart. This device relies on the use of mechanical force to drive the sharp tip through the septum.

Alternatively, radiofrequency perforation apparatuses have been developed, whereby the septal perforation is accomplished by the application of focused radiofrequency energy to the septal tissue via an electrode at the distal end of a relatively thin conductive probe.

Against this background, there exists a continuing need in the industry to provide improved radiofrequency perforation devices and methods. An object of the present invention is therefore to provide such a radiofrequency perforation apparatus.

### SUMMARY

In Example 1, a transseptal access system includes a radiofrequency perforation device having a proximal portion having a proximal portion length, and a distal portion having a distal portion length, the proximal and distal portion lengths defining a perforation device length, wherein the proximal portion has an electrically insulated outer surface, and wherein the distal portion is electrically conductive and terminates at a functional tip, and wherein at least part of the distal portion is uninsulated. The transseptal access system also includes a tubular dilator comprising an elongated body having a body length, a proximal end and a distal end portion terminating in a distal tip, a hub at the proximal end of the body and having a hub length, a dilator lumen extending through the hub and the body and being dimensioned to slidingly receive the radiofrequency perforation device, and an electrical contact element disposed within the hub adjacent to the dilator lumen, wherein the body length and the hub length together define a dilator length; wherein the perforation device length is greater than the dilator length, and wherein the proximal portion length and the distal portion length are dimensioned such that when the radiofrequency perforation device is at a first position within the dilator lumen with the functional tip adjacent to or extending distally from the distal tip of the dilator, no portion of the distal portion of the radiofrequency perforation device extends proximally of the hub; and wherein the proximal portion length and the distal portion length are further dimensioned such when the radiofrequency perforation device is in the first position the electrical contact element is in electrical engagement with the distal portion of the radiofrequency perforation device.

Example 2 is the transseptal access system of Example 1 wherein the proximal portion and the distal portion of the radiofrequency perforation device are substantially isodiametric.

Example 3 is the transseptal access system of Example 1 wherein the dilator lumen has a dilator lumen diameter, and the electrical contact element has a flexible contact member defining an electrical contact element inner diameter that is smaller than the dilator lumen diameter such that the contact member extends partially into the dilator lumen.

Example 4 is the transseptal access system of Example 3 wherein the contact member is deformable so as to maintain electrical engagement with the distal portion of the radiofrequency perforation device when the radiofrequency perforation device is in the first position.

Example 5 is the transseptal access system of Example 1 wherein the proximal portion of the radiofrequency perforation device comprises an electrical insulating material forming the electrically insulated outer surface.

Example 6 is the transseptal access system of any of Examples 1-5 wherein the proximal portion of the radiofrequency perforation device is formed entirely of the electrical insulating material.

Example 7 is the transseptal access system of Example 1 wherein the proximal portion and distal portion of the radiofrequency perforation device are mechanically joined to one another.

Example 8 is the transseptal access system of Example 1 wherein the radiofrequency perforation device comprises an elongated electrically conductive core extending through the proximal and distal portions thereof, the conductive core having a first diameter in the proximal portion and a second diameter greater than the first diameter in the distal portion, and wherein the electrical insulating material is disposed over the core in the proximal portion as an insulative outer layer.

Example 9 is the transseptal access system of Example 1 wherein the proximal portion of the radiofrequency perforation device is configured to be manipulated by a user during operation of the system.

Example 10 is the transseptal access system of any of Examples 1-9 wherein the distal portion of the radiofrequency perforation device has a preformed distal end portion configured to assume an atraumatic shape when sufficiently extended beyond the distal tip of the dilator.

Example 11 is the transseptal access system of Example 10 wherein the atraumatic shape is a J-shape or a pigtail shape.

In Example 12, the transseptal access system of any of Examples 1-11, further comprising an introducer sheath having a sheath lumen configured to receive the dilator for deployment of the dilator at a target tissue site.

In Example 13, the transseptal access system of any of Examples 1-12, further comprising a radiofrequency generator configured to be operatively coupled to the dilator.

Example 14 is the transseptal access system of Example 13 wherein the dilator further comprises a connection port and an electrical lead coupling the electrical contact element and the connection port, wherein the connection port is configured to electrically couple the electrical contact element to the radiofrequency generator.

Example 15 is the transseptal access system of any of Examples 1-14 wherein when the radiofrequency perforation device is in the first position, the radiofrequency generator is configured to deliver radiofrequency energy to the functional tip.

In Example 16, a transseptal access system includes a radiofrequency perforation device having a proximal portion having a proximal portion length, and a distal portion having a distal portion length, the proximal and distal portion lengths defining a perforation device length, wherein the proximal portion has an electrically insulated outer surface, and wherein the distal portion is electrically conductive and terminates at a functional tip, and wherein at least part of the distal portion is uninsulated. The transseptal access system also includes a tubular dilator comprising an elongated body having a body length, a proximal end and a distal end portion terminating in a distal tip, a hub at the proximal end of the body and having a hub length, a dilator lumen extending through the hub and the body and being dimensioned to slidingly receive the radiofrequency perforation device, and an electrical contact element disposed within the hub adjacent to the dilator lumen, wherein the body length and the hub length together define a dilator length; wherein the perforation device length is greater than the dilator length, and wherein the proximal portion length and the distal portion length are dimensioned such that when the radiofrequency perforation device is at a first position within the dilator lumen with the functional tip adjacent to or extending distally from the distal tip of the dilator, no portion of the distal portion of the radiofrequency perforation device extends proximally of the hub; and wherein the proximal portion length and the distal portion length are further dimensioned such when the radiofrequency perforation device is in the first position the electrical contact element is in electrical engagement with the distal portion of the radiofrequency perforation device.

Example 17 is the transseptal access system of Example 16 wherein the proximal portion and the distal portion of the radiofrequency perforation device are substantially isodiametric.

Example 18 is the transseptal access system of Example 16 wherein the dilator lumen has a dilator lumen diameter, and the electrical contact element has a flexible contact member defining an electrical contact element inner diameter that is smaller than the dilator lumen diameter such that the contact member extends partially into the dilator lumen.

Example 19 is the transseptal access system of Example 18 wherein the contact member is deformable so as to maintain electrical engagement with the distal portion of the radiofrequency perforation device when the radiofrequency perforation device is in the first position.

Example 20 is the transseptal access system of Example 16 wherein the proximal portion of the radiofrequency perforation device comprises an electrical insulating material forming the electrically insulated outer surface, and wherein proximal portion of the radiofrequency perforation device is formed entirely of the electrical insulating material.

Example 21 is the transseptal access system of Example 16 wherein the proximal and distal portions of the radiofrequency perforation device are mechanically joined to one another.

Example 22 is the transseptal access system of Example 16 wherein the radiofrequency perforation device comprises an elongated electrically conductive core extending through the proximal and distal portions thereof, the conductive core having a first diameter in the proximal portion and a second diameter greater than the first diameter in the distal portion, and wherein the electrical insulating material is disposed over the core in the proximal portion as an insulative outer layer.

Example 23 is the transseptal access system of Example 16 wherein the proximal portion of the radiofrequency perforation device is configured to be manipulated by a user during operation of the system.

Example 24 is the transseptal access system of Example 16 wherein the distal portion of the radiofrequency perforation device has a preformed distal end portion configured to assume an atraumatic shape when sufficiently extended beyond the distal tip of the dilator.

Example 25 is the transseptal access system of Example 24 wherein the atraumatic shape is a J-shape or a pigtail shape.

In Example 26, the transseptal access system of Example 25, further comprising an introducer sheath having a sheath lumen configured to receive the dilator for deployment of the dilator at a target tissue site.

In Example 27, the transseptal access system of Example 26, further comprising a radiofrequency generator configured to be operatively coupled to the dilator.

Example 28 is the transseptal access system of Example 16 wherein the dilator further comprises a connection port and an electrical lead coupling the electrical contact element and the connection port, wherein the connection port is configured to electrically couple the electrical contact element to the radiofrequency generator.

Example 29 is the transseptal access system of Example 16 wherein when the radiofrequency perforation device is in the first position, the radiofrequency generator is configured to deliver radiofrequency energy to the functional tip.

In Example 30, a transseptal access system assembly using radiofrequency energy includes a radiofrequency perforation device having a proximal portion having a proximal portion length, and a distal portion having a distal portion length. The transseptal access system further includes a tabulator dilator comprising an elongated body having a proximal end and a distal end portion terminating in a distal tip, a hub at the proximal end of the body, a dilator lumen extending through the hub and the body and being dimensioned to slidingly receive the radiofrequency perforation device, and an electrical contact element disposed within the hub adjacent to the dilator lumen, the electrical contact element having a flexible contact member; wherein the perforation device length is greater than the dilator length; and wherein the proximal and distal portions of the radiofrequency perforation device mechanically joined to one another.

Example 31 is the transseptal access system of Example 30 wherein the proximal portion length and the distal portion length are dimensioned such that when the radiofrequency perforation device is at a first position within the dilator lumen with the functional tip adjacent to or extending distally from the distal tip of the dilator, no portion of the distal portion of the radiofrequency perforation device extends proximally of the hub.

Example 32 is the transseptal access system of Example 31 wherein the proximal portion length and the distal portion length are further dimensioned such when the radiofrequency perforation device is in the first position the electrical contact element is in electrical engagement with the distal portion of the radiofrequency perforation device.

Example 33 is the transseptal access system of Example 30 wherein the proximal portion of the radiofrequency perforation device is configured to be manipulated by a user during operation of the system.

In Example 34 a method of making a transseptal access system assembly includes providing a tubular dilator comprising an elongated body having a body length, a proximal end and a distal end portion terminating in a distal tip, a hub at the proximal end of the body and having a hub length, a dilator lumen extending through the hub and the body and being dimensioned to slidingly receive a radiofrequency perforation device, and an electrical contact element disposed within the hub adjacent to the dilator lumen, wherein the body length and the hub length together define a dilator length. The method of making a transseptal access system assembly further includes advancing into the dilator lumen a radiofrequency perforation device having a proximal portion having a proximal portion length, and a distal portion having a distal portion length, the proximal and distal portion lengths defining a perforation device length, wherein the proximal portion has an electrically insulated outer surface, and wherein the distal portion is electrically conductive and terminates at a functional tip, and wherein at least part of the distal portion is uninsulated; wherein the perforation device length is greater than the dilator length, and wherein the proximal portion length and the distal portion length are dimensioned such that when the radiofrequency perforation device is at a first position within the dilator lumen with the functional tip adjacent to or extending distally from the distal tip of the dilator, no portion of the distal portion of the radiofrequency perforation device extends proximally of the hub; and wherein the proximal portion length and the distal portion length are further dimensioned such when the radiofrequency perforation device is in the first position the electrical contact element is in electrical engagement with the distal portion of the radiofrequency perforation device.

In Example 35, the method of Example 34, further comprising an introducer sheath having a sheath lumen configured to receive the dilator for deployment of the dilator at a target tissue site.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIGS. 1A-1C are schematic illustrations of a medical procedure within a patient's heart utilizing a transseptal access system according to embodiments of the present disclosure.

FIGS. 2A-2C are schematic illustrations of a dilator and radiofrequency perforation device of the transseptal access system illustrated in FIGS. 1A-1C, according to embodiments of the present disclosure.

FIGS. 3A and 3B are schematic illustrations of an exemplary electrical contact element, according to embodiments of the present disclosure.

FIG. 4 is a schematic cross-sectional illustration of an exemplary radiofrequency perforation device, according to additional exemplary embodiments of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

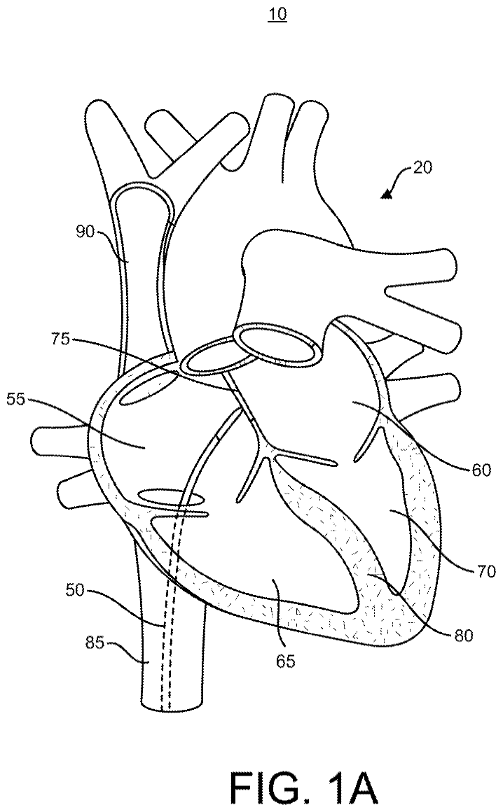

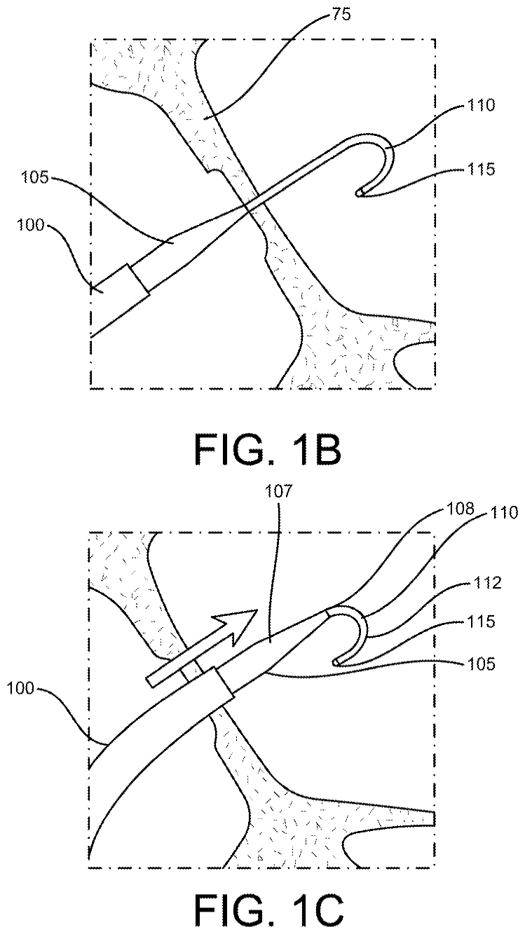

FIGS. 1A-1C are schematic illustrations of a medical procedure 10 within a patient's heart 20 utilizing a transseptal access system 50 according to embodiments of the disclosure. As is known, the human heart 20 has four chambers, a right atrium 55, a left atrium 60, a right ventricle 65 and a left ventricle 70. Separating the right atrium 55 and the left atrium 60 is an atrial septum 75, and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

The medical procedure 10 illustrated in FIGS. 1A-1C is an exemplary embodiment for providing access to the left atrium 60 using the transseptal access system 50 for subsequent deployment of the aforementioned diagnostic and/or therapeutic devices within the left atrium 60. As shown in FIGS. 1A-1C, target tissue site can be defined by tissue on the atrial septum 75. In the illustrated embodiment, the target site is accessed via the IVC 85, for example through the femoral vein, according to conventional catheterization techniques. In other embodiments, access to the target site on the atrial septum 75 may be accomplished using a superior approach wherein the transseptal access system 50 is advanced into the right atrium 55 via the SVC 90.

In the illustrated embodiment, the transseptal access system 50 includes an introducer sheath 100, a dilator 105 having a dilator body 107 and a tapered distal tip portion 108, and a radiofrequency (RF) perforation device 110 having distal end portion 112 terminating in a tip electrode 115. As shown, in the assembled use state illustrated in FIGS. 1A-1C, the RF perforation device 110 can be disposed within the dilator 105, which itself can be disposed within the sheath 100. In one embodiment in which the transseptal access system 50 is deployed into the right atrium 55 via the IVC 85, a user introduces a guidewire (not shown) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator 105 may then be introduced into the sheath 100 and over the guidewire, and advanced through the sheath 100 into the SVC 90. Alternatively, the dilator 105 may be fully inserted into the sheath 100 prior to entering the body, and both may be advanced simultaneously towards the heart 20. When the guidewire, sheath 100 and dilator 105 have been positioned in the SVC 90, the guidewire is removed from the body, and the sheath 100 and the dilator 105 are retracted so that their distal ends are positioned in the right atrium 55. The RF perforation device 110 described can then be introduced into the dilator 105, and advanced toward the heart 20. In certain embodiments, the dilator may be introduced into the body without a need for the sheath 100.

Subsequently, the user may position the distal end of the dilator 105 against the atrial septum 75, which can be done under imaging guidance. The RF perforation device 110 is then positioned such that the tip electrode 115 is aligned with or protruding slightly from the distal end of the dilator 105. The dilator 105 and the RF perforation device 110 may be dragged along the atrial septum 75 and positioned, for example against the fossa ovalis of the atrial septum 75 under imaging guidance. A variety of additional steps may be performed, such as measuring one or more properties of the target site, for example an electrogram or ECG (electrocardiogram) tracing and/or a pressure measurement, or delivering material to the target site, for example delivering a contrast agent. Such steps may facilitate the localization of the tip electrode 115 at the desired target site. In addition, tactile feedback provided by medical RF perforation device 110 is usable to facilitate positioning of the tip electrode 115 at the desired target site.

With the tip electrode 115 and dilator 105 positioned at the target site, energy is delivered from an energy source, e.g., an RF generator, through the RF perforation device 110 to the tip electrode 115 and the target site. In some embodiments, the energy is delivered at a power of at least about 5 W at a voltage of at least about 75 V (peak-to-peak), and functions to vaporize cells in the vicinity of the tip electrode 115, thereby creating a void or perforation through the tissue at the target site. The user then applies force to the RF perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 1 second and about 5 seconds.

With the tip electrode 115 of the RF perforation device 110 having crossed the atrial septum 75, the dilator 105 can be advanced forward, with the tapered distal tip portion 108 operating to gradually enlarge the perforation to permit advancement of the distal end of the sheath 100 into the left atrium 60.

In some embodiments, the distal end portion 112 of the RF perforation device 110 may be pre-formed to assume an atraumatic shape such as a J-shape (as shown in FIGS. 1B-1C), a pigtail shape or other shape selected to direct the tip electrode 115 away from the endocardial surfaces of the left atrium 60. Examples of such RF perforation devices can be found, for example, in U.S. patent application Ser. Nos. 16/445,790 and 16/346,404 assigned to Baylis Medical Company, Inc. The aforementioned pre-formed shapes can advantageously function to minimize the risk of unintended contact between the tip electrode 115 and tissue within the left atrium 60, and can also operate to anchor the distal end portion 112 within the left atrium 60 during subsequent procedural steps. For example, in embodiments, the RF perforation device 110 can be structurally configured to function as a delivery rail for deployment of a relatively larger bore therapy delivery sheath and associated dilator(s). In such embodiments, the dilator 105 and the sheath 100 are withdrawn following deployment of the distal end portion 112 of the RF perforation device 110 into the left atrium 60. The anchoring function of the pre-formed distal end portion 112 inhibits unintended retraction of the distal end portion 112, and corresponding loss of access to the perforated site on the atrial septum 75, during such withdrawal.

The present disclosure describes novel devices and methods for providing safe transseptal access to the left atrium 60 using radiofrequency energy. As will be explained in greater detail herein, the embodiments of the present disclosure simplify the means of providing electrical connectivity between the radiofrequency puncture device and radiofrequency energy generator, while providing enhanced manipulability by the user.

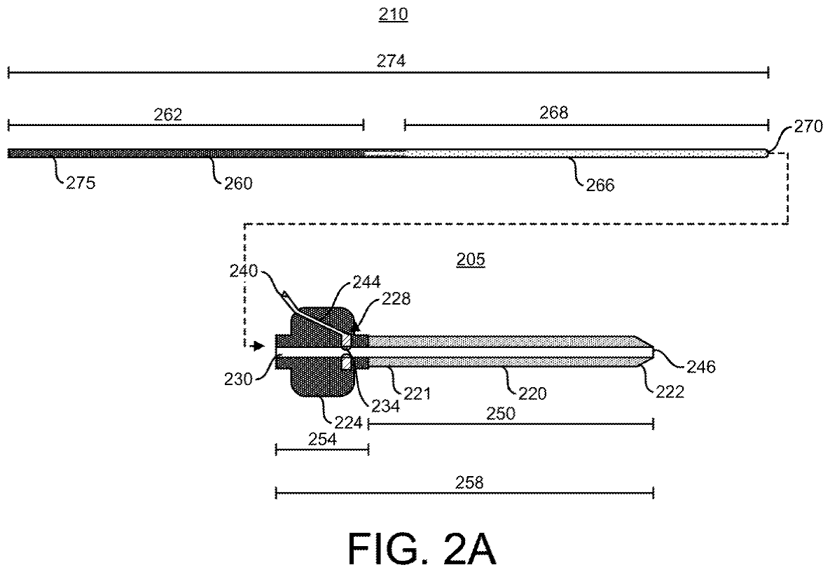

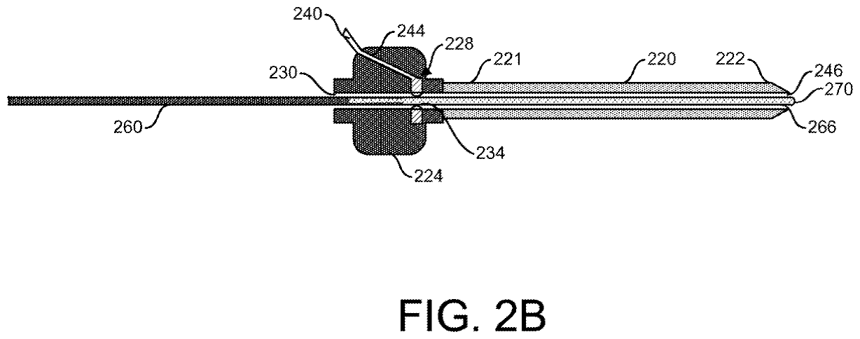

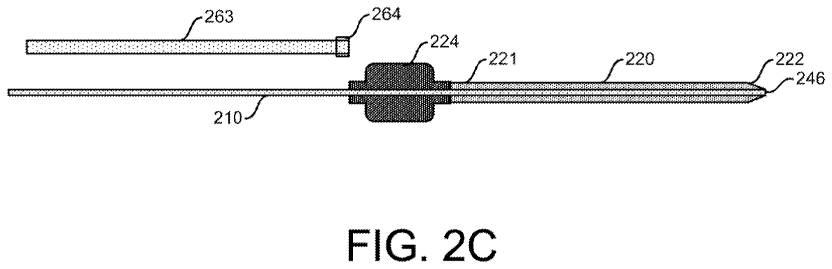

FIGS. 2A-2C are schematic illustrations of a dilator 205 and an RF perforation device 210 according to an embodiment of the present disclosure. As shown, the dilator 205 includes a dilator body 220, a dilator hub 224, an electrical contact element 228 and a dilator lumen 230 extending longitudinally through the hub 224 and the dilator body 220. Additionally, the electrical contact element 228 is positioned within the hub 224 surrounding the dilator lumen 230 and includes a flexible electrical contact member 234 that extends partially into the dilator lumen 230, such that a spacing between inner extremities of the electrical contact member 234 define a contact member inner diameter that is smaller than an inner diameter of the dilator lumen 230.

In addition, the dilator 205 further includes a connection port 240 and an electrical lead 244 coupling the electrical contact element 228 to the connection port 240. In embodiments, the connection port 240 is configured to electrically couple the electrical contact element 228 and consequently, the electrical contact member 234, to a radiofrequency generator (not shown) capable of generating and delivering radiofrequency energy to the electrical contact member 234, as will be explained in greater detail herein.

As further shown, the dilator body 220 has a proximal end 221 and an opposite distal end portion 222 terminating in a distal tip 246. The dilator body 220 has a dilator body length 250. Additionally, the hub 224 is attached to the proximal end 221 of the dilator body 220 and has a hub length 254. The hub length 254 and the dilator body length 250 together define an overall dilator length 258.

As can be further seen from FIGS. 2A-2B, the RF perforation device 210 has a proximal portion 260 having a proximal portion length 262, and a distal portion 266 extending from the proximal portion 260 and having a distal portion length 268 and terminating in a distal functional tip 270 (e.g., a tip electrode such as described above in connection with FIGS. 1A-1C). The proximal and distal portion lengths 262, 268, along with the center mechanical connection of the proximal portion 260 with the distal portion 266, define an overall perforation device length 274. As will be appreciated, the perforation device length 274 is greater than the dilator length 258 so that part of the proximal portion 260 of the RF perforation device 210 extends proximally of the hub 224 when the distal portion 266, particularly the functional tip 270, extends distally of the dilator 205, thus allowing the proximal portion 260 to be manipulated by the user as needed.

In embodiments, the proximal portion 260 of the RF perforation device 210 has an electrically insulated outer surface 275. As such, the proximal portion 260 can be handled directly by the user when the RF perforation device 210 is energized. In the illustrated embodiment, the proximal portion 260 is of a unitary construction formed entirely of an electrically insulative material. One exemplary class of materials for construction of the proximal portion can include various grades of polytetrafluoroethylene (PTFE), polyetheretherketone (PEEK), among others. In embodiments, the proximal portion 260 can further include reinforcing elements, e.g., a polymeric braid or coil, to enhance the structural properties, e.g., stiffness, torque transfer capability, and the like.

In the illustrated embodiment, the distal portion 266 is electrically conductive and is capable of transferring radiofrequency energy supplied by an external RF generator to the functional tip 270 for subsequent delivery to the target tissue in a transseptal crossing procedure, as described above. Any biocompatible electrically conductive material may be selected for construction of the distal portion 266. Exemplary materials may include stainless steel, nickel-titanium alloy, and the like. Further, for ease of illustration, the distal portion 266 is depicted in FIGS. 2A and 2B as a single solid structure, although the skilled artisan will recognize that the construction of the distal portion 266 can vary to accommodate the particular structural requirements for the RF perforation device 210. For example, in embodiments, the distal portion 266 can be constructed as a solid rod, a tube or a coil.

Additionally, in embodiments, the distal portion 266 can be constructed in multiple segments, e.g., a solid rod or hypotube in the regions nearest the proximal portion 260, and a coiled structure more distally to provide enhanced flexibility and torqueability. In embodiments, the distal portion can have a composite construction, e.g., a solid or tubular core conductor surrounded by a wire coil. Additionally in the illustrated embodiment, the proximal and distal portions 260, 266 are substantially isodiametric, although as explained elsewhere herein, this is not a strict requirement in all embodiments.

FIG. 2B schematically illustrates the RF perforation device 210 disposed in an exemplary energized position within the dilator lumen 230. As used herein, “energized position” refers to a position in which part of the distal portion 266 is disposed adjacent to the electrical contact element 228 within the hub 224, with the proximal portion 260 extending proximally from the hub 224 and the functional tip 270 disposed at least near or distally of the distal tip 246 of the dilator body 220. In embodiments, the distal portion 266 has an outer diameter greater than the electrical contact member 234 inner diameter such that when the RF perforation device 210 is in the energized position as shown in FIG. 2B, the electrical contact member 234 electrically engages an outer surface of the conductive distal portion 266. The illustrated engagement of the electrical contact member 234 and the distal portion 266 provides electrical continuity between the functional tip 270 and the connection port 240 and, consequently, to a radiofrequency generator when coupled to the connection port 240 for delivery of radiofrequency energy to the functional tip 270.

As further illustrated, only the proximal portion 260, and no portion of the distal portion 266, of the RF perforation device 210 extends proximally of the hub 224 when the RF perforation device 210 is in the energized position illustrated in FIG. 2B. In other words, when the distal portion 266 of RF perforation device 210 is energized (or capable of being energized) by an RF generator, the user is only exposed to the insulative proximal portion 260, which can be manipulated by the user to perform the desired perforation procedure. Additionally, the energized (or energizable) distal portion 266 is substantially insulated from the external environment by the polymeric dilator body 220 and hub 224, except for the distal-most region of the distal portion 266 and the functional tip 270, the latter of which must necessarily be able to contact the target tissue to be perforated. In embodiments, ideally only about 1 millimeter of the distal-most region of the distal portion 266 and the functional tip 270 will protrude out of the dilator body 220.

As will also be apparent to the skilled artisan, further distal advancement of the RF perforation device 210 will result in the insulative proximal portion 260 being positioned adjacent to the electrical contact element 228, with the distal portion 266 positioned distally of the electrical contact element 228. When so positioned, electrical continuity will no longer exist between the distal portion 266, the functional tip 270 and the connection port 240. This has the advantage of preventing unintended and unnecessary delivery of radiofrequency energy to the functional tip 270 once the perforation step has been completed and the functional tip 270 is located within the left atrium 60.

In embodiments, as illustrated in FIG. 2C, the RF perforation device 210 may include further temporary insulation. An extra sheath 263 may be placed over the RF perforation device 210 proximal to the dilator hub 224. The extra sheath 263 provides further protection to the user by preventing accidental contact with the uninsulated portion of the RF perforation device 210 while radiofrequency is being applied to the system in its energized position. The extra sheath 263 could slide over, snap over, or be built into the system. The extra sheath 263 can further include reinforcing elements like a torque interface 264 to provide enhanced torqueability. Any biocompatible insulative sterilizable plastics or rubbers may be selected for construction of the extra sheath 263. Exemplary materials may include a thin plastic material that would allow the user to grab and manipulate the RF perforation device 210 through the sheath, to facilitate positioning and advancing of the RF perforation device 210.

In view of the foregoing, the respective lengths of the components of the dilator 205 (i.e., the dilator length 258, the dilator body length 250 and the hub length 254) and the RF perforation device 210 (i.e., the proximal portion length 262 and the distal portion length 268) are defined so as to accomplish the desired selective electrical connectivity between the RF generator and the functional tip 270 of the RF perforation device 210 based on the positions of the RF perforation device 210 within the dilator lumen 230, while at the same time ensuring that only the insulative proximal portion 260 of the RF perforation device 210 is exposed to the user when such connectivity is made. In various embodiments, with a dilator length 258 of between 60 and 130 centimeters, the proximal portion length 262 and the distal portion length 268 may be between 75 and 250 centimeters and 60 and 130 centimeters, respectively. In one embodiment, the dilator body length 250 may be 85 centimeters, the proximal portion length 262 may be 160 centimeters, and the distal portion length 268 may be 87 centimeters. Still other ranges of the respective lengths may be utilized within the scope of the disclosure depending on the particular clinical needs.

For ease of illustration, the RF perforation device 210 is shown in FIGS. 2A-2C as being substantially isodiametric, but this is not critical. Rather, the diameter of the proximal and/or distal portions 260, 266 may vary along their respective lengths depending on the particular functional requirements for a given procedure. For example, portions of the proximal portion 260 may have an increased diameter relative to other portions to, among other things, provide enhanced manipulability by the user and/or to delimit the degree to which the RF perforation device 210 can be advanced distally within the dilator lumen 230. Still additionally, the distal-most regions of the distal portion 266 may have a diameter that is greater or less than the more proximal regions so as to tailor the stiffness and columnar strength of the respective regions. The only strict requirement is that a region of the distal portion 266 defining a contact region thereof must have a diameter greater than the inner diameter of the electrical contact member 234 to ensure the desired electrical connectivity between the functional tip 270 and an external RF generator.

Furthermore, the dilator 205 and/or the RF perforation device 210 may include additional features to enhance their functionality and usability. By way of example, one or both of the dilator body 220 and the distal portion 266 of the RF perforation device 210 may include imaging markers (e.g., radiopaque or echogenic structures) at selected locations to enhance the visibility of the devices under imaging modalities. As another example, fiducial markers may be included along the proximal portion 260 of the RF perforation device 210 to provide the user with a visual indication of the relative positions of the RF perforation device 210 and the dilator 205. Still other value-added features may be utilized to enhance the usability of the system 50.

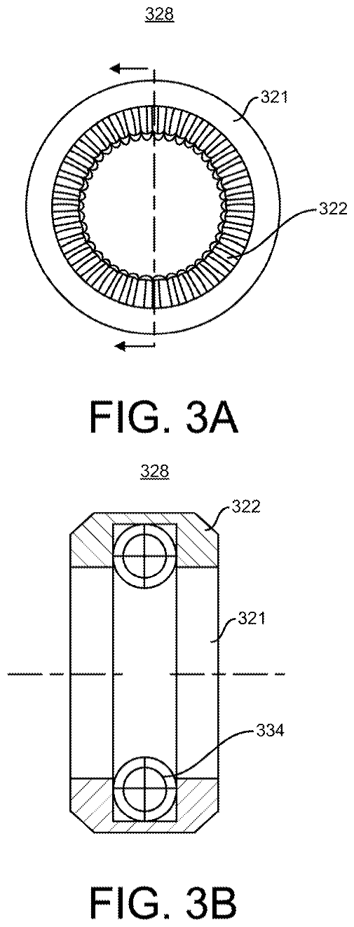

FIGS. 3A and 3B are schematic illustrations of an exemplary electrical contact element 328 of the dilator 205 according to embodiments of the present disclosure. The electrical contact element 328 may be substantially structurally and functionally identical to the electrical contact element 228 of FIGS. 2A-2C.

As shown, the electrical contact element 328 includes an outer portion 321 made of any electrically conductive metal, like stainless steel, and an inner portion 322 made of spring coils, with the diameter of the outer portion 321 being larger than the inner diameter of the inner portion 322. As illustrated in FIG. 3B, the inner portion 322 creates a housing for a flexible electrical contact member 334, corresponding to the electrical contact member 234 of FIGS. 2A and 2B. In embodiments, the housing is typically cylindrical, but the geometry can be customized.

As described, the flexible electrical contact member 334 extends partially into the dilator lumen 230, such that a spacing between the inner extremities of the electrical contact member 334 define a contact member inner diameter that is smaller than the inner diameter of the dilator lumen 230. In embodiments, the electrical contact member 334 electrically engages an outer surface of the conductive distal portion 266, shown in FIG. 2B. The electrical contact member 334 has an inner diameter less than the diameter of the distal portion 266 of the RF perforation device 210 to ensure the desired electrical connectivity between the functional tip 270 and an external RF generator (not shown). The engagement between the electrical contact member 334 and the distal portion 266 provides electrical continuity between the functional tip 270 and the connection port 240 and, consequently, to a radiofrequency generator when coupled to the connection port 240 for delivery of radiofrequency energy to the functional tip 270.

In embodiments, the electrical contact element 328 is a bi-directional axial spring design that enables low contact resistance without the need for noble metals. In embodiments, the electrical contact element 328 is the Bal Conn® for IS-1 cardiac applications from Bal Seal Engineering, LLC. In embodiments, the electrical contact element 328 has a lead diameter of 2.67 mm, a breakout force max of 3.10 N, running force of between 0.50 N to 0.90 N, and a static dry contact resistant of 100 mΩ (nominal), +/−60 mΩ (tolerance).

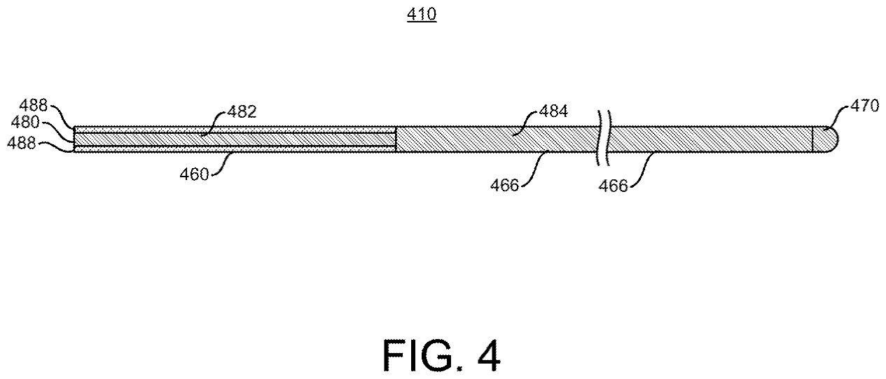

FIG. 4 is a schematic cross-sectional illustration of an alternative RF perforation device 410 according to embodiments of the disclosure. The RF perforation device 410 may be substantially structurally and functionally identical to the RF perforation device 210, except as described in connection with FIG. 4.

As shown, the RF perforation device 410 includes a proximal portion 460, a distal portion 466 and a functional tip 470. The RF perforation device 410 further includes a conductive core 480 extending along the lengths of the proximal portion 460 and the distal portion 466. The core 480 has a proximal section 482 corresponding to the proximal portion 460, and a distal section 484 corresponding to the distal portion 466 of the RF perforation device 410. As shown, the diameter of the proximal section 482 is smaller than the diameter of the distal section 484, and an insulating layer 488 is disposed over the proximal section 482 to define the insulative outer surface of the proximal section 460 that can be handled by the user when the RF perforation device is in an energized position within the dilator. The conductive core 480 may be a unitary construction or may be comprised of separate proximal and distal sections 482, 484 joined together by mechanical joining methods (e.g., welding, brazing, and the like).

For ease of illustration only the distal portions 266, 466 of the RF perforation devices 210, 410 are shown to be uninsulated. In embodiments, however, these portions may be selectively insulated, with a region remaining uninsulated so as to provide the desired selective electrical engagement with an RF generator, as described elsewhere herein.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

I claim:

1. A transseptal access system comprising:
a radiofrequency perforation device having a proximal portion having a proximal portion length, and a distal portion having a distal portion length, the proximal and distal portion lengths defining a perforation device length, wherein the proximal portion has an electrically insulated outer surface, and wherein the distal portion is electrically conductive and terminates at a functional tip, and wherein at least part of the distal portion is uninsulated; and
a tubular dilator comprising an elongated body having a body length, a proximal end and a distal end portion terminating in a distal tip, a hub at the proximal end of the body and having a hub length, a dilator lumen extending through the hub and the body and being dimensioned to slidingly receive the radiofrequency perforation device, and an electrical contact element disposed within the hub adjacent to the dilator lumen, wherein the body length and the hub length together define a dilator length,
wherein the perforation device length is greater than the dilator length, and wherein the proximal portion length and the distal portion length are dimensioned such that when the radiofrequency perforation device is at a first position within the dilator lumen with the functional tip adjacent to or extending distally from the distal tip of the dilator, no portion of the distal portion of the radiofrequency perforation device extends proximally of the hub, and
wherein the proximal portion length and the distal portion length are further dimensioned such when the radiofrequency perforation device is in the first position the electrical contact element is in electrical engagement with the distal portion of the radiofrequency perforation device.

2. The transseptal access system of claim 1, wherein the proximal portion and the distal portion of the radiofrequency perforation device are substantially isodiametric.

3. The transseptal access system of claim 1, wherein the dilator lumen has a dilator lumen diameter, and the electrical contact element has a flexible contact member defining an electrical contact element inner diameter that is smaller than the dilator lumen diameter such that the contact member extends partially into the dilator lumen.

4. The transseptal access system of claim 3, wherein the contact member is deformable so as to maintain electrical engagement with the distal portion of the radiofrequency perforation device when the radiofrequency perforation device is in the first position.

5. The transseptal access system of claim 1, wherein the proximal portion of the radiofrequency perforation device comprises an electrical insulating material forming the electrically insulated outer surface, and wherein proximal portion of the radiofrequency perforation device is formed entirely of the electrical insulating material.

6. The transseptal access system of claim 1, wherein the proximal and distal portions of the radiofrequency perforation device are mechanically joined to one another.

7. The transseptal access system of claim 1, wherein the radiofrequency perforation device comprises an elongated electrically conductive core extending through the proximal and distal portions thereof, the conductive core having a first diameter in the proximal portion and a second diameter greater than the first diameter in the distal portion, and wherein the electrical insulating material is disposed over the core in the proximal portion as an insulative outer layer.

8. The transseptal access system of claim 1, wherein the proximal portion of the radiofrequency perforation device is configured to be manipulated by a user during operation of the system.

9. The transseptal access system of claim 1, wherein the distal portion of the radiofrequency perforation device has a preformed distal end portion configured to assume an atraumatic shape when sufficiently extended beyond the distal tip of the dilator.

10. The transseptal access system of claim 9, wherein the atraumatic shape is a J-shape or a pigtail shape.

11. The transseptal access system of claim 10, further comprising an introducer sheath having a sheath lumen configured to receive the dilator for deployment of the dilator at a target tissue site.

12. The transseptal access system of claim 11, further comprising a radiofrequency generator configured to be operatively coupled to the dilator.

13. The transseptal access system of claim 1, wherein the dilator further comprises a connection port and an electrical lead coupling the electrical contact element and the connection port, wherein the connection port is configured to electrically couple the electrical contact element to the radiofrequency generator.

14. The transseptal access system of claim 1, wherein when the radiofrequency perforation device is in the first position, the radiofrequency generator is configured to deliver radiofrequency energy to the functional tip.

15. A transseptal access system assembly using radiofrequency energy, the transseptal access system comprising:
a radiofrequency perforation device having a proximal portion having a proximal portion length, and a distal portion having a distal portion length, and
a tabulator dilator comprising an elongated body having a proximal end and a distal end portion terminating in a distal tip, a hub at the proximal end of the body, a dilator lumen extending through the hub and the body and being dimensioned to slidingly receive the radiofrequency perforation device, and an electrical contact element disposed within the hub adjacent to the dilator lumen, the electrical contact element having a flexible contact member,
wherein the perforation device length is greater than the dilator length, and
wherein the proximal and distal portions of the radiofrequency perforation device mechanically joined to one another.

16. The transseptal access system of claim 15, wherein the proximal portion length and the distal portion length are dimensioned such that when the radiofrequency perforation device is at a first position within the dilator lumen with the functional tip adjacent to or extending distally from the distal tip of the dilator, no portion of the distal portion of the radiofrequency perforation device extends proximally of the hub.

17. The transseptal access system of claim 16, wherein the proximal portion length and the distal portion length are further dimensioned such when the radiofrequency perforation device is in the first position the electrical contact element is in electrical engagement with the distal portion of the radiofrequency perforation device.

18. The transseptal access system of claim 15, wherein the proximal portion of the radiofrequency perforation device is configured to be manipulated by a user during operation of the system.

19. A method of making a transseptal access system assembly, the method comprising:
providing a tubular dilator comprising an elongated body having a body length, a proximal end and a distal end portion terminating in a distal tip, a hub at the proximal end of the body and having a hub length, a dilator lumen extending through the hub and the body and being dimensioned to slidingly receive a radiofrequency perforation device, and an electrical contact element disposed within the hub adjacent to the dilator lumen, wherein the body length and the hub length together define a dilator length, and
advancing into the dilator lumen a radiofrequency perforation device having a proximal portion having a proximal portion length, and a distal portion having a distal portion length, the proximal and distal portion lengths defining a perforation device length, wherein the proximal portion has an electrically insulated outer surface, and wherein the distal portion is electrically conductive and terminates at a functional tip, and wherein at least part of the distal portion is uninsulated,
wherein the perforation device length is greater than the dilator length, and wherein the proximal portion length and the distal portion length are dimensioned such that when the radiofrequency perforation device is at a first position within the dilator lumen with the functional tip adjacent to or extending distally from the distal tip of the dilator, no portion of the distal portion of the radiofrequency perforation device extends proximally of the hub, and
wherein the proximal portion length and the distal portion length are further dimensioned such when the radiofrequency perforation device is in the first position the electrical contact element is in electrical engagement with the distal portion of the radiofrequency perforation device.

20. The method of claim 19, further comprising an introducer sheath having a sheath lumen configured to receive the dilator for deployment of the dilator at a target tissue site.
